(19) United States
(12) Patent Application Publication
(10) Pub. No.: US 2020/0365045 A1
(43) Pub. Date: Nov. 19, 2020

(54) PACKAGE FOR SELLING CONDOM PRODUCTS AND USAGE METHOD THEREOF

(71) Applicant: Seçkin Güngör, Clifton, NJ (US)

(72) Inventor: Seçkin Güngör, Clifton, NJ (US)

(21) Appl. No.: 16/414,697

(22) Filed: May 16, 2019

(51) Int. Cl. G09B 5/02; G06K 7/14; G06K 7/10; A61F 6/00; A61F 6/04

(52) U.S. Cl. CPC G09B 5/02; G06K 7/1417; G06F 16/9558; A61F 6/005; A61F 6/04; G06K 7/10297

(57) ABSTRACT

The current invention relates to a system and technique of packaging and use of condom products, as a primary means of protection method. The invention is particularly related to a condom package that varies sexual life by means of presenting a code with the condom, attaching this code with a smart device, reaching a link through a smart device, determining a type of instruction by clicking on the link to a browser based or non-browser based application, presenting the information unique to the determined type, and is related to a usage method thereof.

### FIELD OF INVENTION

The current invention relates to a system and technique of packaging and use of the condom products, as a primary means of protection method.

The invention is particularly related to a condom package that varies sexual life by means of presenting a code with the condom, attaching this code with a smart device, reaching a link through a smart device, determining a type of instruction by clicking on the link to a browser based or non-browser based application, presenting the information unique to the determined type, and is related to a usage method thereof.

### BACKGROUND

Research conducted in different communities worldwide have shown that most people have uniform sexual lives. That eventually bring them boredom, dissatisfaction and depression along with a variety of similar other odds, which, in most cases, entail to separation of married couples.

Various products have been introduced and marketed across the globe for making sexual life more dynamic and exciting. Yet, most pose a threat to human health.

Use of condoms is one of the clinically approved and most recognized and favored methods of sexual protection. Manufacturers popularize the use of condoms by diversifying and making minor to major modifications in their production ranges, in an effort to fortify their marketing power, further.

The condoms used in the current art are generally formed in one type. The Production-Development studies made on the condoms concentrate on the production quality, technical differences and aroma issues.

The condoms brought to use by the current state of the art are typical outputs of canonical production. Which hardly have any role to play in terms of altering sexual life, or, making it exuberant.

As a result, for solving the abovementioned problems in the current art, the requirement of a condom package and the usage method thereof that is fun and will make sexual life dynamic and exciting along with the insufficiency of the solutions, make it necessary to make a development in the relevant technical field.

### SUMMARY

The aim of the present invention is to help people eliminate the cynical implications of such feelings of boredom, unhappiness, depression etc. by altering their accustomed or routinised patterns of sexual relation.

Another aim of the present invention is to make sexual life more dynamic, joyful and exciting for both, or, all partners.

Another aim of the present invention is to create public awareness by means of the system and method presented. So that rates of community-wide adoption and use of condoms can be increased, to prevent incidents of sexually transmitted diseases.

Another aim of the invention is to adapt the educational and instructional power of the technology to the sexual life by means of mobile communication devices.

Another aim of the invention is both to provide sexual protection and to inform in terms of a more fun and varied sexual relationship.

Another aim of the invention is to provide information that enriches the sexual life by describing instructional sexual techniques and show different positions after the code provided by the condom is transferred to the smart device.

A condom package used in an instructional system with a smart device to enhance condom use is disclosed in which a code is provided on the condom package and presented to the smart device. The smart device reads the code and directs the user to a link to a browser application or non-browser application. Upon clicking the link, to execute the browser or non-browser application, a type selection section is displayed on the smart device for determining a type of instruction. The type is randomly determined and the smart device is configured to then present information of an education and application unique to the determined type to the user.

These features, advantages and other embodiments of the present invention are further made apparent, in the remainder of the present document, to those of ordinary skill in the art.

### BRIEF DESCRIPTION OF THE DRAWINGS

The exemplary aspects of the present invention will be understood more fully from the detailed description given below and from the accompanying drawings of various embodiments of the invention, which, however, should not be taken to limit the invention to the specific embodiments, but are for explanation and understanding only.

FIG. 1 illustrates the condom package according to an embodiment of the present invention.

FIG. 2 shows reading of the condom package by a smart device to initiate a usage method, according to an embodiment of the present invention.

FIG. 3A shows the screen of the smart device after activation, opening an URL link to display a type selection section, through a web browser application or application, according to an embodiment of the present invention.

FIG. 3B shows the screen of the smart device after the type is selected, for example a video, according to an embodiment of the present invention.

FIG. 3C shows the screen of the smart device further displaying an educational animation video, according to an embodiment of the present invention.

FIG. 4 shows a flowchart of the condom package usage method according to an embodiment of the present invention.

### REFERENCE NUMBERS

10 Condom Package
11 Entrance Code
20 Smart Device
21 Type selection Section
400 Method of using condom package
402-412 Method Steps

The invention will be understood more clearly with the above mentioned reference numbers and described with reference to the attached figures.

### DETAILED DESCRIPTION

The purpose of the following detailed description is to provide an understanding of one or more embodiments of the present invention. Those of ordinary skills in the art will realize that the following detailed description is illustrative only and is not intended to be in any way limiting. Other embodiments will readily suggest themselves to such skilled persons having the benefit of this disclosure and/or description.

Various embodiments of the present invention illustrated in the drawings may not be drawn to scale. Rather, the dimensions of the various features may be expanded or reduced for clarity. In addition, some of the drawings may be simplified for clarity. Thus, the drawings may not depict all of the components of a given apparatus (e.g., device) or method. The same reference indicators will be used throughout the drawings and the following detailed description to refer to the same or like parts.

In accordance with the embodiment(s) of present invention, the components, process steps, and/or data structures described herein may be implemented using various types of operating systems, computing platforms, computer programs, and/or general-purpose machines. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Erasable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card and paper tape, and the like) and other known types of program memory.

The term “system” or “device” including “smart device” is used generically herein to describe any number of components, elements, sub-systems, devices, packet switch elements, packet switches, access switches, routers, networks, computer and/or communication devices or mechanisms, or combinations of components thereof. The term “computer” includes a processor, memory, and buses capable of executing instruction wherein the computer refers to one or a cluster of computers, personal computers, workstations, mainframes, or combinations of computers thereof.

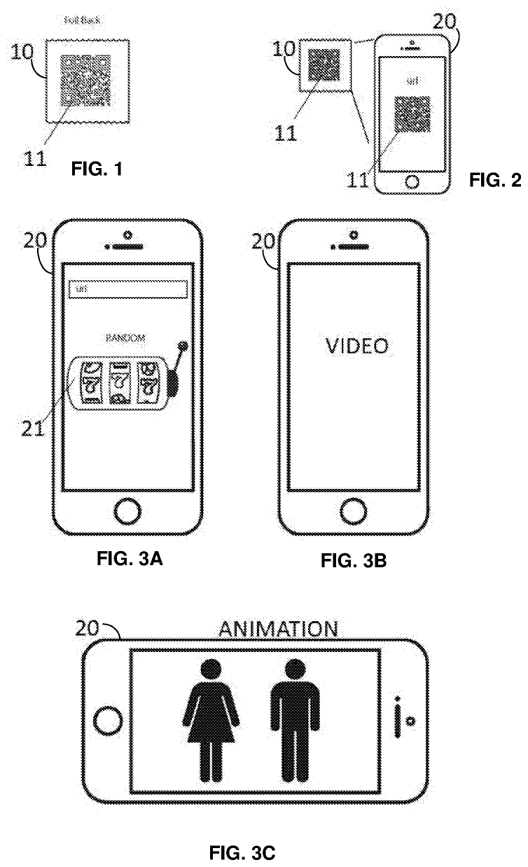

As shown in FIG. 1, an entrance code 11 such as QR code, password, RFID (Radio Frequency Identification) or NFC (Near Field Communication) compatible data etc. is provided with a condom package 10. The code 11 is activated by means of the smart device 20 which is configured to read the code 11 as shown in FIG. 2. An URL link is obtained after the code is activated through the smart device. See FIG. 3A. When the URL link is activated, the smart device connects to the site and the type selection section (21) is displayed that is used randomly to make a selection of type of instruction. In other instances, after the code is activated, instead of obtaining an URL which executes a web browser or non-browser based application, a link to access/install an application is obtained. The type selection area (21) is started online and randomly a type is selected. The educational story, visual, video and animation unique to each predetermined types are transmitted to display on the smart device. See FIG. 3B. By means of this educational data, the user reads an appropriate story of that type and has a sexual relationship/experience by applying the information in the displayed visual, video or animation. See FIG. 3C.

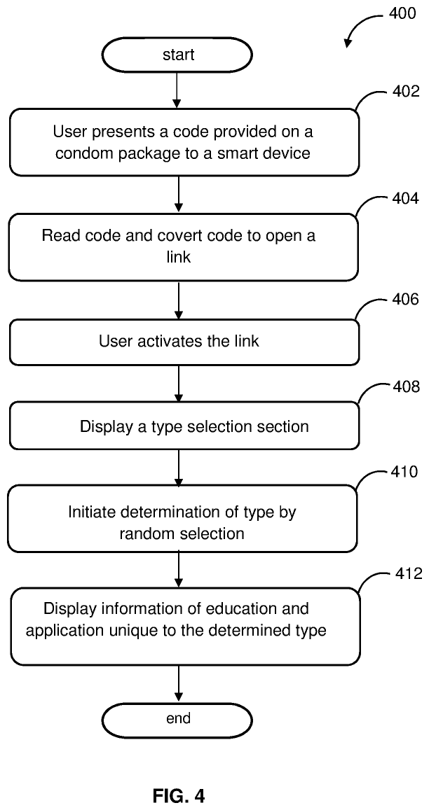

In a particular embodiment of the present invention, FIG. 4 is a flowchart 400 illustrating the method of using the condom package in the instructional system with a smart device. A QR code 11 is created on the condom package 10 as provided in FIGS. 1-2 for illustrative purposes. At block 402, the code on the condom package is presented to a smart device such a smart mobile device 20. At block 404, the QR code 11 is read by the smart phone 20 and the user is directed to the link to a browser based or non-browser based application. At block 406 the user activates the link by clicking on the link to the browser based or non-browser based application. When the user clicks on the link, as shown at block 408, the type selection section 21, in other words, the slot machine, appears in which types are entered and the educational data such as the story, visual, video and animation are loaded. At block 410, the handle of the slot machine is turned and randomly a type is determined. At block 412, the relevant educational and application based story, visual description or animation in the name of this type is transmitted to the user. The user applies this information and application qualified by this type with his/her partner. Therefore, a monotonous sexual life is diversified.

While particular embodiments of the present invention have been shown and described, it will be obvious to those skilled in the art that, based upon the teachings herein, changes and modifications may be made without departing from the exemplary embodiments of the present invention and its broader aspects. Therefore, the appended claims are intended to encompass within their scope all such changes and modifications as are within the true spirit and scope of this exemplary embodiments of the present invention.

What is claimed is:

1. A condom package used in an instructional system with a smart device to enhance condom use, comprising:
a code provided on the condom package;
the smart device configured to read the code and the code configured to direct
the smart device to present a link on a display of the smart device;
upon activating the link, a type selection section displayed on the smart device for determining a type of instruction;
upon determining the type of instruction, hereinafter the determined type, the
smart device configured to present information of an education and application unique to the determined type to the user.

2. The condom package used in an instructional system with a smart device according to claim 1, wherein the code is configured to direct the smart device to present a link on a display of the smart device to execute a web browser application.

3. The condom package used in an instructional system with a smart device according to claim 1, wherein the code is configured to direct the smart device to present a link on a display of the smart device to execute a non browser based application.

4. The condom package used in an instructional system with a smart device according to claim 1, wherein the code is configured to direct the smart device to present a link on a display of the smart device to install an application.

5. The condom package used in an instructional system with a smart device according to claim 1, wherein the code provided by said condom package is a QR code.

6. The condom package used in an instructional system with a smart device according to claim 1, wherein the code provided by said condom package is a password.

7. The condom package used in an instructional system with a smart device according to claim 1, wherein the code provided by said condom package is created according to NFC (Near Field Communication) technology.

8. The condom package used in an instructional system with a smart device according to claim 1, wherein the code provided by said condom package is created according to RFID (Radio Frequency Identification) technology.

9. The condom package used in an instructional system with a smart device according to claim 1, wherein the type of instruction is determined randomly.

10. The condom package used in an instructional system with a smart device according to claim 1, wherein the education and application unique to the determined type are presented in the form of a story.

11. The condom package used in an instructional system with a smart device according to claim 1, wherein the education and application unique to the determined type are presented in a visual manner.

12. The condom package used in an instructional system with a smart device according to claim 1, wherein the education and application unique to the determined type are presented in the form of video.

13. The condom package used in an instructional system with a smart device according to claim 1, wherein the education and application unique to the determined type are presented in the form of an animation.

14. A method of using a condom package in an instructional system with a smart device, the method comprising the steps of:
a user presenting a code provided by the condom package to the smart device;
activating the code with the smart device by the smart device reading the code;
the user determining a type selection of instruction, hereinafter, the determined type;
the smart device presenting information of an education and application unique to the determined type to the user.

15. The method according to claim 14, wherein upon reading the code, the smart device presenting a link on a display of the smart device and the user clicking the link to execute a web browser application prior to determining the determined type.

16. The method according to claim 14, wherein upon reading the code, the smart device presenting a link on a display of the smart device and the user clicking the link to execute a non browser based application prior to determining the determined type.

17. The method according to claim 14, wherein upon reading the code, the smart device presenting a link on a display of the smart device and the user clicking the link to install an application prior to determining the determined type.

18. The method according to claim 14, wherein the code provided by said condom package is a QR code.

19. The method according to claim 14, wherein the education and application unique to the determined type are presented in the form of a story.

20. The method according to claim 14, wherein the education and application unique to the determined type are presented in the form of an animation.
